(19) United States
(12) Patent Application Publication
(10) Pub. No.: US 2012/0129178 A1
(43) Pub. Date: May 24, 2012

(54) GENOMIC POLYMORPHISM FOR PREDICTING THERAPEUTIC RESPONSE

(75) Inventors: Heinz-Josef Lenz, Altadena, CA (US); Sheeja Thankappan Pullarkat, Glendale, CA (US); Yi Ping Xiong, Alhambra, CA (US)

(73) Assignee: University of Southern California

(21) Appl. No.: 13/153,233

(22) Filed: Jun. 3, 2011

Related U.S. Application Data
(63) Continuation of application No. 12/110,044, filed on Apr. 25, 2008, now abandoned.
Application No. 12/110,044 is a continuation of application No. 09/715,764, filed on Nov. 15, 2000, now abandoned.
(60) Provisional application No. 60/165,574, filed on Nov. 15, 1999.

(51) Int. Cl. C12Q 1/68; G01N 27/447

(52) U.S. Cl. 435/6.12; 435/6.13; 204/450

(57) ABSTRACT

The present invention relates to the use of genomic polymorphism to provide individualized therapeutic regimens to treat patients suffering from diseases such as cancer. The invention discloses methods for determining the efficacy or choice of chemotherapeutic drugs and regimens for use in treating a diseased patient by associating genomic polymorphism with the effectiveness of the drugs or regimens, or by associating genomic polymorphism with the intratumoral expression of a gene whereby the gene expression affects effectiveness of the drugs or regimens. In particular, the present invention provides novel methods for screening therapeutic regimens, which comprise determining a patient's genotype at a tandemly repeated 28 base pair region in the thymidilate synthase (TS) gene's 5′ untranslated region (UTR). Patients homozygous for a triple repeat will be least successfully treated with a thymidylate synthase directed drug, while those heterozygous for a triple and a double repeat will be more successfully treated, and those homozygous for a double repeat will be even more successfully treated. Those patients homozygous for the double repeat will likely suffer the least side effects from thymidylate synthase directed drugs such as 5-FU.

### RELATED APPLICATIONS

This invention claims priority to U.S. Provisional Application Ser. No. 60/165,574, filed Nov. 15, 1999.

### FIELD OF THE INVENTION

This invention relates to the field of pharmacogenomics and specifically to the application of genomic polymorphism to treat diseases.

### BACKGROUND OF THE INVENTION

In nature, organisms of the same species usually differ in some aspects of their appearance. The differences are genetically determined and are referred to as polymorphism. At many gene loci, two or more alleles may occur (genetic polymorphism). Genetic polymorphism is defined as the occurrence in a population of two or more genetically determined alternative phenotypes due to different alleles. Polymorphism can be observed at the level of the whole individual (phenotype), in variant forms of proteins and blood group substances (biochemical polymorphism), morphological features of chromosomes (chromosomal polymorphism) or at the level of DNA in differences of nucleotides (DNA polymorphism).

Amongst the various types of DNA polymorphism is polymorphism that results from allelic differences in the number of repeats at a given locus. This type of polymorphism has been called variable number of tandem repeat (VNTR) polymorphism. There are three possible genotypes with respect to two alleles at any one locus: (1) homozygous for one allele, (2) heterozygous for the two alleles and (3) homozygous for the other allele. For example, in VTR polymorphism, a genomic sample can be homozygous for a triple repeat allele, homozygous for a double repeat allele or heterozygous for a double and a triple repeat allele.

Although, the relationship between an individual's capacity to metabolize environmental carcinogens and other xenobiotics and susceptibility to cancer has been extensively studied (17-20), the role that polymorphism may play in determining individual differences in the response to drugs has not been studied. There is a great need for such studies, though. For example, cancer chemotherapy is limited by significant inter-individual variations in responses and toxicities, which may be due to genetic alterations in drug metabolizing enzymes or receptor expression. Thus, pharmacogenetic screening prior to anticancer drug administration may lead to identification of specific populations predisposed to drug toxicity or poor drug response (16).

The significance of pharmacogenetics (the effect of genetic differences on drug response) in cancer chemotherapy is further underlined by the fact that:
1. Target ligands may be heterogeneous with respect to amount or structure;
2. Many genes are targets of prodrugs and these genes are involved in the biotransformation of active compounds by enzymes that exhibit genetic polymorphisms;
3. Certain anticancer drugs are detoxified by polymorphic enzyme systems;
4. Most cancer drugs have significant inter-patient variability in pharmacokinetics and toxicity.

Thus, the use of polymorphism can fulfill the great need for improved methods of prognosis and treatment guidelines for treating cancer, a need which is dramatically exemplified by the fact that current concepts and clinical practice regarding the prognosis and the therapy for patients with adenocarcinomas of the large bowel rest on clinical/pathological staging which has stood for over 60 years. Unquestionably, then, methods for rapidly and easily identifying individuals likely to benefit from chemotherapy and those likely to experience side effects are greatly needed. Also, methods to determine appropriate dosing levels for patients are needed.

### SUMMARY OF THE INVENTION

The present invention relates to the use of genomic polymorphism to provide individualized therapeutic regimens to treat patients suffering from diseases such as cancer. The invention discloses methods for determining the efficacy or choice of chemotherapeutic drugs and regimens for use in treating a diseased patient by associating genomic polymorphism with the effectiveness of the drugs or regimens, or by associating genomic polymorphism with the intratumoral expression of a gene whereby the gene expression affects effectiveness of the drugs or regimens. In particular, the present invention provides novel methods for screening therapeutic regimens, which comprise determining a patient's genotype at a 28 base pair region in the thymidilate synthase (TS) gene's 5′ untranslated region (UTR).

### BRIEF DESCRIPTION OF THE DRAWINGS

FIG. 1 shows the results of electrophoresis of PCR products on 4% agarose gel. The FIGURE shows single 220 bp and 250 bp base-pair bands for the S/S and L/L homozygotes, respectively. The FIGURE shows double bands for the heterozygotes.

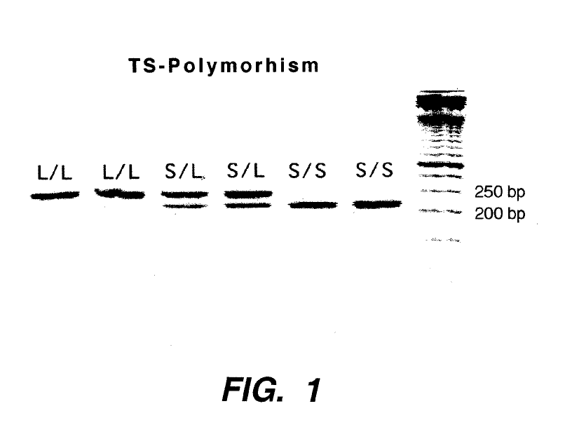

### DETAILED DESCRIPTION OF THE PREFERRED EMBODIMENTS

### Definitions

For convenience, the meaning of certain terms and phrases employed in the specification, examples, and appended claims are provided below.

The term “5′ UTR” refers to the 5′ untranslated region of the thymidilate synthase (TS) gene, located near the initiation start site.

The term “allele”, which is used interchangeably herein with “allelic variant” refers to alternative forms of a gene or portions thereof. Alleles occupy the same locus or position on homologous chromosomes. Alleles of a specific gene can differ from each other in a single nucleotide, or several nucleotides, and can include substitutions, deletions, and insertions of nucleotides. An allele of a gene can also be a form of a gene containing a mutation.

As used herein, the term “nucleic acid” refers to polynucleotides such as deoxyribonucleic acid (DNA), and, where appropriate, ribonucleic acid (RNA). The term should also be understood to include, as equivalents, derivatives, variants and analogs of either RNA or DNA made from nucleotide analogs, and, as applicable to the embodiment being described, single (sense or antisense) and double-stranded polynucleotides. Deoxyribonucleotides include deoxyadenosine, deoxycytidine, deoxyguanosine, and deoxythymidine. For purposes of clarity, when referring herein to a nucleotide of a nucleic acid, which can be DNA or an RNA, the terms “adenosine”, “cytidine”, “guanosine”, and thymidine” are used. It is understood that if the nucleic acid is RNA, a nucleotide having a uracil base is uridine.

The term “nucleotide sequence complementary to the nucleotide sequence set forth in SEQ ID NO: x” refers to the nucleotide sequence of the complementary strand of a nucleic acid strand having SEQ ID NO: x. The term “complementary strand” is used herein interchangeably with the term “complement”. The complement of a nucleic acid strand can be the complement of a coding strand or the complement of a non-coding strand. When referring to double stranded nucleic acids, the complement of a nucleic acid having SEQ ID NO: x refers to the complementary strand of the strand having SEQ ID NO: x or to any nucleic acid having the nucleotide sequence of the complementary strand of SEQ ID NO: x. When referring to a single stranded nucleic acid having the nucleotide sequence SEQ ID NO: x, the complement of this nucleic acid is a nucleic acid having a nucleotide sequence which is complementary to that of SEQ ID NO: x. The nucleotide sequences and complementary sequences thereof are always given in the 5′ to 3′ direction. The term “complement” and “reverse complement” are used interchangeably herein.

The term “polymorphism” refers to the coexistence of more than one form of a gene or portion thereof. A portion of a gene of which there are at least two different forms, i.e., two different nucleotide sequences, is referred to as a “polymorphic region of a gene”. A polymorphic region can be a single nucleotide, the identity of which differs in different alleles. A polymorphic region can also be several nucleotides long.

A “polymorphic gene” refers to a gene having at least one polymorphic region.

The term “TS directed drug” refers to drugs that involve or are targeted against or are based on thymidilate synthase.

### Predictive Medicine and Pharmacogenomics

A powerful association has been discovered between a patient's genotype and her response to chemotherapy drugs. In the general case, the invention establishes for the first time that polymorphisms of genes involved with the target of anticancer drugs and metabolism of anti-cancer drugs may be predictive of intratumoral gene expression levels. Polymorphism profiles can, thus, be used to determine the selection or dosing of chemotherapeutic drugs. The results of the examples of the invention also help explain the differences in toxicities and efficacy of anticancer drugs in different ethnic groups because most of these polymorphisms have been shown to have ethnic group associated characteristic gene frequencies.

In particular, an association has been discovered between the variable number of tandem repeats polymorphism (also referred to as “genomic polymorphism” or “TS polymorphism” herein) in the 5′ untranslated region (5′ UTR) of the TS gene of a subject and the response of the subject to TS directed drug therapy. This association provides the basis for a convenient, reliable a priori method to determine whether TS directed drug therapy will be effective in treating the subject. This will ensure that patients who will not respond to TS directed drug therapy do not suffer the unnecessary side effects associated with such therapy.

Patients homozygous for a double repeat of the tandemly repeated sequence will be most successfully treated with a TS directed drug, while patients heterozygous for a triple repeat and a double repeat will be less successfully treated with such a drug, and patients homozygous for a triple repeat will be least successfully treated with the drug. TS directed drugs include, but are not limited to, fluoropyrimidines such as 5-fluorouracil.

Thymidylate synthase (TS) is the enzyme that catalyzes the intracellular methylation of deoxyuridine-5′-monophosphate (dUMP) to thymidine-5′-monophosphate (dTMP) (4). This reaction is the sole de novo source of thymidylate, which is an essential precursor for DNA synthesis. TS is also the critical target enzyme for many chemotherapeutic drugs. For example, 5-Fluorouracil (5-FU) is a TS directed chemotherapeutic agent belonging to the class of fluoropyrimidines, which are widely used in the treatment of malignancies in the gastrointestinal, breast, and upper aerodigestive tract (5). The active metabolite of 5-FU, 5-fluorodeoxyuridylate (5-FdUMP) binds to TS and inhibits the conversion of deoxyuridine 5′ monophosphate (dUMP) to deoxythymidine 5′-monophosphate (dTMP) by forming a stable covalent ternary complex. This results in the depletion of cellular thymidylate pools and cessation of DNA synthesis (6). Therefore, sensitivity or resistance to 5-FU is dependent on levels of TS in the tumors (7). It has been shown that a tandemly repeated sequence, present in the 5′ UTR downstream from the cap-site in the 5′-terminal regulatory region modulates hTS gene expression (8). This sequence is a cis-acting enhancer element and is polymorphic, containing either a double or triple repeat of a 28 base pair sequence (9).

As the examples below illustrate, it is possible to predict TS gene expression in a tumor by measuring the TS polymorphism in peripheral blood cells. Because TS gene expression determines the effectiveness of TS directed drugs, identification of TS polymorphism allows one to decide whether a TS directed drug, e.g., 5-FU, will have benefit but also may determine the risk of side effects of treatment with such drugs. Thus, for the first time, TS polymorphism could allow to individualize the dose and choice of an anticancer drug.

Thus, in a preferred embodiment, the invention provides a method for determining the effectiveness of a therapeutic regimen in the treatment of a cancer in a subject, which method comprises (a) determining a genomic polymorphism in the subject with the cancer; and (b) correlating the efficacy of the therapeutic regimen with the type of genomic polymorphism exhibited by the subject. In one embodiment, the therapeutic regimen comprises administering a chemotherapeutic drug to the subject. Such a drug, for example, is a fluropyrimidine. In one embodiment, the fluoropyrimidine is 5-fluorouracil. In a preferred embodiment, the subject is a human subject.

In one embodiment of the above methods, determining the genomic polymorphism of the subject comprises determining the subject's genotype at a tandemly repeated 28 base pair sequence in the thymidilate synthase gene's 5′ UTR whereby the subject will exhibit the poorest response to administration of a TS directed drug, e.g., 5-fluorouracil, if the subject's genotype is homozygous for a triple repeat of the tandemly repeated sequence, a less poor response to administration of the same TS directed drug (e.g., 5-fluorouracil) if the subject's genotype is heterozygous for a double repeat and a triple repeat of the tandemly repeated sequence, and the best response to administration of the TS directed drug (e.g., 5-fluorouracil) if the subject's genotype is homozygous for a double repeat of the tandemly repeated sequence.

In a preferred embodiment, determining the subject's genotype further comprises: extracting genomic DNA from a biological sample of the subject; amplifying the 5′ UTR of the thymidilate synthase gene of said genomic DNA using polymerase chain reaction; and analyzing the polymerase chain reaction product to determine the subject's genotype. According to some embodiments, the analysis of the polymerase chain reaction product is performed using electrophoresis.

In various embodiments, the invention provides a method for determining the effectiveness of a therapeutic regimen in the treatment of various cancers including, but not limited to, colorectal cancer, gastric cancer, breast cancer, Burkitt's lymphoma, B follicular cell lymphoma, small cell lung carcinoma and esophageal cancer.

The invention also provides for a method for predicting the effect of a therapeutic regimen for treating a cancer in a human subject wherein a chemotherapeutic drug is administered to the human, which method comprises associating a genomic polymorphism of the human subject with intratumoral expression of a gene wherein said gene expression influences the efficacy of said therapeutic regimen. In a preferred embodiment, the gene is thymidilate synthase gene and the drug is a drug that targets thymidilate synthase, e.g., a fluoropyrimidine. In a preferred embodiment of this method, the genomic polymorphism of the human subject is the subject's genotype at a tandemly repeated 28 base pair sequence in the thymidilate synthase gene's 5′ UTR. In such a method, the therapeutic regimen is most effective if the subject's genotype is homozygous for a double repeat of the tandemly repeated sequence, is less effective if the subject's genotype is heterozygous for a double and a triple repeat of the tandemly repeated sequence and is least effective if the subject's genotype is homozygous for a triple repeat of the tandemly repeated sequence.

Further, the invention provides a method for determining the expression level of a gene in cells of a subject, the method comprising determining a genomic polymorphism of the subject; and associating the expression level of said gene with said genomic polymorphism. The gene is thymidilate synthase gene in one embodiment of this method. In another embodiment of this method, the genomic polymorphism of the subject is the subject's genotype at a tandemly repeated 28 base pair sequence in the thymidilate synthase gene's 5′ UTR. The expression level of the TS gene is highest if the subject's genotype is homozygous for a triple repeat of the tandemly repeated sequence, is less if the subject's genotype is heterozygous for a double and a triple repeat of the tandemly repeated sequence and is least if the subject's genotype is homozygous for a double repeat of the tandemly repeated sequence.

The invention also provides a method for determining the effectiveness of a chemotherapeutic regimen wherein a TS directed drug such as a fluoropyrimidine is administered to a human subject, the method comprising: determining the subject's genotype at a tandemly repeated 28 base pair sequence in the thymidilate synthase gene's 5′ U whereby the subject will exhibit the poorest response to administration of the TS directed drug if the subject's genotype is homozygous for a triple repeat of the tandemly repeated sequence, a less poor response to administration of the TS directed drug if the subject's genotype is heterozygous for a double repeat and a triple repeat of the tandemly repeated sequence, and the best response to administration of the TS directed drug if the subject's genotype is homozygous for a double repeat of the tandemly repeated sequence. As noted above, this method can be practiced with fluoropyrimidines, e.g., 5-fluorouracil.

In the general case, the invention provides a method for determining an appropriate chemotherapeutic regimen to treat a cancer in a subject, the method comprising: associating a genomic polymorphism of the subject with the effectiveness of a chemotherapeutic regimen. This method, for example, is used to select or reject a chemotherapeutic drug to treat the cancer.

In order to accomplish the identification of individuals or tissue samples within the scope of the present invention, a tissue sample is obtained. It will be appreciated that the sample may comprise any type of tissue. For most applications, it is likely that blood would be the tissue of choice. This would be true in the case of paternity testing and the like. However, other tissues, including skin, semen, hair, and other body fluids or tissues may be acceptable for specific purposes. Using the methods of the present invention, no more than approximately 10 .μl of blood is required in order to perform the testing procedure. DNA can be obtained from any nucleated cell that is live, dead, or preserved.

Detecting which genomic polymorphism is present in the subject's sample may be accomplished by determining the defining characteristic of the genomic polymorphism that the genomic DNA of the subject possesses. As one of the ordinary skill in the art would know, there are many means and methods available to make such a determination, e.g., electrophoresis, automated sequencing, allele-specific oligonucleotide probing, differential restriction endonuclease digestion, ligase-mediated gene detection, and the like.

The testing procedure, for example, requires that the cells in the tissue sample be lysed and that the DNA obtained from the lysed cells be isolated and cleaved with a restriction enzyme. It should be appreciated that because the variability at a VNTR locus arises from copy number differences of tandem repeats, any restriction endonuclease with sites flanking the repeats will reveal the polymorphism. The enzymes noted in the specification are representative and are non-limiting examples of enzymes which can be used for a VTR clone. In a preferred embodiment, restriction enzymes with sites very close to the cluster of repeats are desired. The result is smaller restriction fragments which are easier to discriminate on agarose gels. The DNA can then be applied to gel and electrophoresed using widely known and generally accepted procedures.

Genomic DNA of a subject can be amplified to make detection of the VNTR polymorphism easier. Amplification of nucleic acid may be achieved using conventional methods, see, e.g., Maniatis, et al., Molecular Cloning: A Laboratory Manual 187-210 (Cold Spring Harbour Laboratory, 1982) which is incorporated herein by reference. Amplification, however, is preferably accomplished via the polymerase chain reaction (“PCR”) method disclosed by U.S. Pat. Nos. 4,698,195 and 4,800,159, the respective contents of which are incorporated herein by reference. Thus, oligonucleotide primer pairs can be constructed that allow enzymatic amplification of a subject's nucleic acid that determines the VNTR polymorphism in the 5′ UTR of the TS gene. The amplified nucleic acid can then be assayed to determine which type of polymorphism is present.

Primer pairs suitable for use in the practice of the present invention are linear oligonucleotides ranging in length from about ten to about thirty nucleotides in length. One of the primers in the pair should be complementary to a nucleotide sequence upstream of the nucleic acid sequence that determines the VNTR polymorphism in the 5′ UTR of the TS gene targeted for amplification. The other primer should be complementary to a sequence located down stream of this target site. The sequences complementary to the primer pairs may be separated by as many nucleotides as the PCR technique and the other technique(s) for detecting the presence or absence of VNTR polymorphism will allow, provided that an appropriate control is used. Primers suitable for use in the practice of the present invention are set forth in the methodology section below.

### Kits

As set forth herein, the invention provides methods, e.g., diagnostic and therapeutic methods, for determining the type of the polymorphic region present in the TS gene. Accordingly, the invention provides kits for practicing these methods.

In a preferred embodiment, the invention provides kits for use in screening for the effectiveness of TS directed drug therapy in human subjects. Such kits can include all or some of the positive controls, negative controls, reagents, primers, sequencing markers, probes and antibodies described herein for determining the presence or absence of the tandem repeat nucleic acid sequences that define the genomic polymorphism in the 5′ UTR of the TS gene. Kits of the present invention may contain, for example, double or triple repeats of the 28 base pair sequence in the 5′ UTR of the TS gene, double and triple repeats of the 28 base pair sequence in the 5′ UTR of the TS gene, schedules of the number and type of nucleotide sequence repeats and characteristics of one or more labeled oligonucleotide probes specific for one or more of the tandem repeat sequences of the VNTR polymorphism, one or more primers for amplification of nucleic acid sequences that determine the VNTR polymorphism in the 5′ UTR of the TS gene, reagents commonly used for amplification, polymerase, and combinations of any of the above.

As amenable, these suggested kit components may be packaged in a manner customary for use by those of skill in the art. For example, these suggested kit components may be provided in solution or as a liquid dispersion or the like.

A presently preferred embodiment of the inventive kits for use in screening for the effectiveness of TS directed drug therapy comprises DNA tandem repeat sequences that determine type of the VNTR polymorphism of the TS gene in Tris-EDTA buffer solution preferably kept at 4.degree. C.

Another embodiment of the inventive kits for use in screening for the effectiveness of TS directed drug therapy further comprises one or more primers specific for amplification of nucleic acid sequences that define the VNTR polymorphism in the 5′ UTR of the TS gene, for example, primers selected from the group comprising SEQ ID NO 1 to SEQ ID NO 7.

Yet another embodiment of the inventive kits for use in screening for the effectiveness of TS directed drug therapy further comprises sequencing markers ranging in size from about 100 to about 600 base pairs.

### EXAMPLES

Data from 58 patients with colorectal cancer with known TS gene expression level was obtained. This data demonstrates a significant correlation between TS polymorphism and intratumoral TS gene expression. This observation is the first time that a genomic polymorphism has been found to associated with intratumoral gene expression levels. In the case of TS this may have a significant impact on patient management, regarding selection of drug and dosing of drug. Patients with triple repeat in the TS gene as expected from in vitro models had higher gene expression levels in their tumors compared to patients with double repeat (p=0.003). 16 patients with the triple repeat had a median TS gene expression of 7.15, 10 patients with the double repeat had a median TS gene expression of 4.04 and 29 patients with heterozygosity (one triple and one double) had a median TS gene expression of 2.38.

These results can be summarized as follows (as used herein, S will refer to a short (double) repeat and L will refer to a long or triple repeat):

Median TS Gene Expression Levels (95%CI)

L/L: 7.15 (4.53, 11.24)

S/L: 4.04 (2.94,5.54)

S/S: 2.38 (1.39, 4.09)

Correlation Between Polymorphism and TS Gene Expression Levels in the Tumor

L/L vs S/L: p=044

L/L vs S/S: p=003

S/L vs S/S: p=0.10

The examples of the present invention show that the polymorphic region affects the TS mRNA levels in both normal and tumor tissues. Individuals homozygous for the triple repeat variant (L/L) had 3.5 times higher TS mRNA levels compared to those homozygous for the double repeat variant (S/S) in tumor tissue (p=0.003). In addition, there was a statistically significant difference between the S/L and the S/S groups (p=0.04). In normal tissues, TS expression was 2.5 times higher in the L/L compared to the S/S group.

Although, it has been known that TS mRNA levels are a determinant of response to fluoropyrimidine based chemotherapy and survival in patients with gastric and colorectal cancers (2,3), the significance of TS polymorphism in determining TS expression has not been previously studied.

The results of the examples herein establish that the polymorphism in the hTS gene affects the TS mRNA levels in tumors and in normal tissue. Genomic DNA was extracted from 52 metastatic liver samples and 26 normal liver samples from patients with advanced colon cancer. Genotyping for the polymorphism was done as described in the methodology section using the polymerase chain reaction to amplify the polymorphic region. Homozygotes for the triple repeat variant designated as (L/L) had a 250 bp product, those homozygous for the double repeat variant (S/S) had 220 bp product and heterozygotes (S/L) had 220 and 250 base pair products. The TS mRNA level was determined by RT-PCR in both the tumor and normal tissue samples as described below.

Of the 52 metastatic liver samples, fifteen (29%) were homozygous (UL) for the triple repeat, twenty-six (50%) were heterozygous (S/L), and eleven (21%) were homozygous (S/S) for the double repeat variant. The mean intra-tumoral TS mRNA expression and the 95% confidence interval (CI) for these three groups were 9.42 (5.51, 16.12) for those with L/L genotype, 5.53 (3.68, 8.31) for heterozygotes and 2.60 (1.39, 4.87) in those with the S/S genotype respectively (Table 1a). The difference in the TS mRNA levels between the L/L and the S/S groups was statistically significant (p=0.003), as was the difference between the S/L and S/S groups (p=0.04) by pair-wise comparison (Table 1b).

The TS mRNA level in 26 normal liver specimens was also examined. Of these, seven patients (27%) had the L/L genotype, fourteen patients (54%) had the heterozygous (S/L) genotype and five patients (19%) had the homozygous S/S genotype. The mean TS mRNA level and 95% CI were 8.21 (4.79, 14.06) for L/L genotype, 4.56 (3.12, 6.68) for the heterozygotes and 3.19 (1.69, 6.03) for the S/S genotype respectively (Table 1a).

The data show for the first time that the number of tandemly repeated sequences in the hTS gene affects the levels of TS mRNA in tumor and normal tissues. The TS mRNA levels in tissues increased with the number of tandem repeats. Individuals with the L/L genotype had 3.5 times higher TS mRNA expression in tumor tissue and about 2.5 times higher in normal tissue when compared with levels in comparable tissues in individuals with the S/S genotype.

While gene expression is not a direct measure of enzyme activity, Curt et al. have shown that when gene amplification takes place, it is closely related to increases in the enzyme and mRNA levels (10). Moreover, in earlier studies in gastric and colon cancer, it has been shown that TS protein levels are closely correlated to the TS mRNA levels within individual tumors.

These results have immense clinical significance owing to the fact that previous studies have shown that TS mRNA levels in tumor tissue in patients with gastric and colon cancer patients predicts both response to chemotherapy with 5-FU (5-Fluorouracil) and survival rate (2,3). In 57 patients with gastric cancer who were evaluable for response to treatment with 5-Fu, it was demonstrated that the difference in the mean TS mRNA levels in responding and resistant patients was statistically significant (p<0.001) by the two-sided Wilcoxon test. The median survival was 43+ months in patients with TS mRNA levels of 54.6 when compared to 6 months in those whose levels were >4.6 (P=0.004) based on the two-sided Pearson chi square test (8). In a separate study of 46 patients with disseminated colon cancer it has been shown that low expression of TS mRNA was associated with a higher probability of response to 5-FU based treatment and survival. The median TS of 3.5 significantly segregated responders from non-responders (P=0.001) based on the two-sided Pearson chi square test. No patient with TS mRNA levels higher than 4.1 responded to treatment (9).

Therefore, genotyping patients for the TS polymorphism prior to chemotherapy with drugs directed against TS, e.g., fluoropyrimidines, has the potential to identify those patients who will respond to such drugs. Non-responders can be subjected to alternative non-TS directed treatment and thus spared the unwanted side effects of drugs like fluoropyrimidines.

Because it has been demonstrated that the tandemly repeated sequence in the hTS gene determines the TS mRNA levels in tissues, based on the number of tandem repeats it can be predicted that patients homozygous for the triple repeat variant are likely to have tumors with high TS expression. As such, they may be expected to be relatively resistant to TS-directed treatment and should be subjected to non-TS directed chemotherapy like the newer chemotherapeutic agent Irinotecan (targets topoisomerase-I), thus sparing them of the toxic side effects of 5-FU. Hence, genotyping patients for the TS polymorphism by the simple method of PCR amplification of genomic DNA, provides the opportunity to optimize TS directed (for example, fluoropyrimidine based) chemotherapy by selecting only those patients whose tumors are likely to respond. These findings also apply to the newer drugs directed against TS such as newer fluoropyrimidine agents like capecitabine and UFT, which are increasingly being used for the treatment of a variety of cancers.

### Methodology

### PCR Quantitation of TS mRNA

The isolation of RNA was based on the method reported by Chomczynski and Sacchi (11). RNA was converted to cDNA using reverse transcriptase and random hexamers. A PCR-based method was used to quantitate the TS gene expression level (12). The expression of the β actin gene was used as an internal standard. In each sample, the linear range of cDNA amplification was established. Relative gene expression was calculated as the ration between the amount of the radiolabeled PCR product with the linear amplification range of the TS gene and the β actin gene. PCR conditions, T7 RNA polymerase transcription and the quantitation procedure are described by Horikoshi et al. (12). Each 5′ primer had the T7 polymerase sequence SEQ ID NO: 1-TAATACGACTCACTTATA attached to its 5′ end which gives 500-fold amplification of the target genes. The primers used were: TS60, SEQ ID NO: 2-GATGTGCGCAATCATGTAACGTGAG, corresponding to bases 697-720 of the TS coding sequence (13); TS61, T7- SEQ ID NO: 3 “GGGAGA”GGAGTTGACCAACTGCAAAGAGTG, corresponding to bases 469-492 of the TS coding sequence (13). The primers for the β actin coding region are BA67: SEQ ID NO: 4-“GGGAGA”GCGGGAAATCGTGCGTGACATT, corresponding to bases 2104 to 2127 of the β actin genomic sequence located in exon 3 (14); and BA68: SEQ ID NO: 5-GATGGAGTTGAAGGTAGMCGTG, corresponding to bases 2409-2432 of the β actin genomic sequence, located in exon 4 (14).

### Analysis of TS Gene Polymorphism

We obtained core-needle biopsy frozen samples from normal and metastatic liver, in patients with disseminated colon cancer. Genomic DNA was extracted using the Qiagen kit (Qiagen, Valencia, Calif.). The 5′ UTR of the hTS gene was amplified by PCR using the following primers: Primer 1 (sense): SEQ ID NO: 6-GTGGCTCCTGCGTTTCCCCC; and Primer 2 (antisense): SEQ ID NO: 7-GCTCCGAGCCGGCCACAGGCATGGCGCGG as previously described (7). 25 μL reaction mixture containing 1.25 mM MgCl₂ was transferred to a thermal cycler (PTC-100™, MJ Research Laboratories) and amplified for 35 cycles. Each cycle consisted of 1 minute at 96° C., 30 seconds at 60° C., and 1 minute at 72° C., with a final extension phase at 72° C. for 5 minutes. The PCR product was analyzed by electrophoresis on a 4% agarose gel. Genotype was indicated by the banding pattern (S/S=220 bp; S/L=220 and 250 bp; and L/L=250 bp).

### Statistical Method

The logarithm was taken prior to the analysis. An analysis of the variance (ANOVA) was performed to test the relationship of the TS expression and TS genes in tissues from those patients with colon cancer using the transformed values of the TS. The analysis was done for the tissues from normal liver and metastatic liver tissue separately. The overall p-values were based on the F-test from the ANOVA. The LSD (least significant difference) method (15) was used for multiple comparison. Paired t-test was used to test the difference of the TS expression among patients with colon cancer between tissues from normal and metastatic liver tissues.

The tables present the geometric mean (after transformation then using the exponential transformation to convert back to the original scale) and the associated 95% confidence intervals to summarize the study data.

TABLE 1a
Correlation between TS genotype and TS gene expression.

| TS | | | | | | |
|---|---|---|---|---|---|---|
| Genotype | Normal Liver Tissue | | | Metastatic Liver Tissue | | |
| genotype | N | TS mean | 95% CI* | N | TS mean | 95% CI* |
| L/L | 7 | 8.21 | (4.79, 14.06) | 15 | 9.42 | (5.51, 16.12) |
| S/L | 14 | 4.56 | (3.12, 6.68) | 26 | 5.53 | (3.68, 8.31) |
| S/S | 5 | 3.19 | (1.69, 6.03) | 11 | 2.6 | (1.39, 4.87) |

Gene and TS expression by tissue.
*95% Confidence Interval.

TABLE 1b
Pairwise comparisons of TS expression by TS genotype
in tumor tissue by TS genes in tumor tissue.

| | |
|---|---|
| TS | Tumor Tissue |

| Genotype | TS Mean | p-value |
|---|---|---|
| overall | | 0.011 |
| L/L vs. S/S | 9.42 vs. 2.60 | 0.003 |
| L/L vs. S/L | 9.42 vs. 5.53 | 0.12 |
| S/L vs. S/S | 5.53 vs. 2.60 | 0.048 |

*The p-value for the overall comparison was based on the F-test, and all other p-vaules were based on the LSD method for multiple comparison.

### REFERENCES

1. Lenz, H. J. et al. Thymidylate Synthase mRNA level in adenocarcinoma of the stomach: A predictor for primary tumor response and overall survival. Journal of Clinical Oncology 14:176-182 (1995).
2. Leichman, C. G. et al. Quantitation of Intratumoral Thymidylate synthase expression predicts for disseminated colorectal cancer response and resistance to protracted-infusion fluorouracil and weekly leucovorin. Journal of Clinical Oncology 15:3223-3229 (1997).
3. Horie, N; Aiba, H; Ogura, K; Hojo, H; and Takeishi, K; Functional Analysis and DNA polymorphism of the tandemly repeated sequences in the 5′-terminal regulatory region of the human gene for thymidylate synthase. Cell Structure and function 20:191-197 (1995).
4. Heidelberger, C; Chandari, N. K; Danenenberg, P. et al. Fluorinated pyrimidines: A new class of tumor inhibitory compounds. Nature 179:663-666 (1957).
5. Moertel, C. G. Chemotherapy for colorectal cancer. N. Engl. J. Med 330:1136-1143 (1994).
6. Danenberg, P. Thymidylate synthase—A target enzyme in cancer chemotherapy. Biochem Biophys Acta 473:73-92 (1977).
7. Santi, D. V; Mc Henry, C. S; Sommer, H. Mechanism of interaction of thymidylate synthetase with 5-fluorodeoxyuridylate. Biochemistry 13:471-481 (1974).
8. Horie, N; Chimoto, M; Nozawa, R; and Takeishi, K; Characterization of the regulatory sequences and nuclear factors that function in cooperation with the promoter of the human thymidylate synthase gene. Biochim. Biophys. Acta 1216:409-416
9. Marsh, S. et al Ethnic variation in the thymidylate synthase enhancer region polymorphism among Caucasian and Asian populations. Genomics 58:1-3 (1999).
10. Curt, G. A. et al; Unstable methtrexate resistance in human small cell carcinomas associated with double minute chromosomes. N Engl J Med 308:199-202 (1983).
11. Chomczynski, P; and Sacchi, N. Single-step method of RNA isolation by acid guanidium. thiocyabate-phenol-chloroform extraction. Anal. Biochem 162:156-159 (1987).
12. Horikoshi, T. et al. Quantitation of thymidylate synthase, dihydrofolate reductase, and DT-diaphorase gene expression in human tumors using the polymerase chain reaction. Cancer Research 52:108-116 (1992).
13. Takeishi, K. et al. Nucleotide sequence of a functional cDNA for thymidylate synthase. Nucleic Acids Res 13:2035-2045 (1987).
14. Ng, S. Y. et al. Evolution of the functional β actin gene and its multipseudogene family; Conservation of non-coding regions and chromosomal dispersion of pseudogenes. Mol Cell Biol 5:2720-2732.(1985).
15. Zar, J. H. Biostatical Analysis. Prentice-Hall, Inc, Englewood Cliffs, N.J 151-155. (1974).
16. Iyer, L. and Ratain, M. J. Eur. J. Cancer 34:1493-9 (1998).
17. Nebert, D. W. et al. Human drug-metabolizing enzyme polymorphisms: effects on risk of toxicity and cancer. DNA Cell Biol. 15:273-80 (1996).
18. Shields, P. G. et al. Pharmacogenetics: detecting sensitive populations. Environ Health Perspect. 102 Suppl 11:81-7 (1994).
19. Caporaso et al. 1991 Study design and genetic susceptibility factors in the risk assessment of chemical carcinogens. Ann. 1st Super Sanita. 27:621-30 (1991).
20. Ikawa, S. et al. Assessment of cancer susceptibility in humans by use of genetic polymorphisms in carcinogen metabolism. Pharmacogenetics 5:S154-60 (1995).

What is claimed is:

1. A method for determining the effectiveness of a therapeutic regimen for the treatment of a cancer in a subject, the method comprising:
(a) determining a genomic polymorphism in the subject with said cancer; and
(b) concluding that the therapeutic regimen will be effective if the genomic polymorphism exhibited by the subject is of a certain type.

2. The method of claim 1 wherein the therapeutic regimen comprises administering a chemotherapeutic drug to the subject.

3. The method of claim 2 wherein the chemotherapeutic drug is a TS directed drug.

4. The method of claim 3 wherein the TS directed drug is a fluropyrimidine.

5. The method of claim 4 wherein the fluoropyrimidine is 5-fluorouracil.

6. The method of claim 5 wherein the subject is a human subject.

7. The method of claim 6 wherein determining the genomic polymorphism of the subject comprises determining the subject's genotype at a tandemly repeated 28 base pair sequence in the thymidilate synthase gene's 5′ UTR whereby the subject will exhibit the poorest response to administration of 5-fluorouracil if the subject's genotype is homozygous for a triple repeat of the tandemly repeated sequence, a less poor response to administration of 5-fluorouracil if the subject's genotype is heterozygous for a double repeat and a triple repeat of the tandemly repeated sequence, and the best response to administration of 5-fluorouracil if the subject's genotype is homozygous for a double repeat of the tandemly repeated sequence.

8. The method of claim 6 wherein determining the subject's genotype further comprises:
extracting genomic DNA from a biological sample of the subject;
amplifying the 5′ UTR of the thymidilate synthase gene of said genomic DNA using polymerase chain reaction; and
analyzing the polymerase chain reaction product to determine the subject's genotype.

9. The method of claim 8 wherein analysis of the polymerase chain reaction product is performed using electrophoresis.

10. The method of claim 1 wherein the cancer is breast cancer.

11. The method of claim 1 wherein the cancer is colorectal cancer.

12. The method of claim 1 wherein the cancer is gastric cancer.

13. The method of claim 1 wherein the cancer is esophageal cancer

14. The method of claim 1 wherein the cancer is Burkitt's lymphoma.

15. The method of claim 1 wherein the cancer is B follicular cell lymphoma.

16. The method of claim 1 wherein the cancer is small cell lung carcinoma.

17. A method for predicting the effect of a therapeutic regimen for treating a cancer in a human subject wherein a chemotherapeutic drug is administered to the human, the method comprising:
associating a genomic polymorphism of the human subject with intratumoral expression of a gene wherein said gene expression influences the efficacy of said therapeutic regimen.

18. The method of claim 17 wherein the chemotherapeutic drug is a TS directed drug.

19. The method of claim 18 wherein the gene is thymidilate synthase gene.

20. The method of claim 19 wherein the genomic polymorphism of the human subject is the subject's genotype at a tandemly repeated 28 base pair sequence in the thymidilate synthase gene 5′ UTR.

21. The method of claim 20 wherein the therapeutic regimen is most effective if the subject's genotype is homozygous for a double repeat of the tandemly repeated sequence, is less effective if the subject's genotype is heterozygous for a double and a triple repeat of the tandemly repeated sequence and is least effective if the subject's genotype is homozygous for a triple repeat of the tandemly repeated sequence.

22. A method for determining the expression level of a gene in cells of a subject, the method comprising:
determining a genomic polymorphism of the subject; and
associating the expression level of said gene with said genomic polymorphism.

23. The method of claim 22 wherein the gene is thymidilate synthase gene.

24. The method of claim 23 wherein the genomic polymorphism of the subject is the subject's genotype at a tandemly repeated 28 base pair sequence in the thymidilate synthase gene's 5′ UTR.

25. The method of claim 24 wherein the expression level of said gene is highest if the subject's genotype is homozygous for a triple repeat of the tandemly repeated sequence, is less if the subject's genotype is heterozygous for a double and a triple repeat of the tandemly repeated sequence and is least if the subject's genotype is homozygous for a double repeat of the tandemly repeated sequence.

26. A method for determining the effectiveness of a chemotherapeutic regimen wherein a TS directed drug is administered to a human subject, the method comprising:
determining the subject's genotype at a tandemly repeated 28 base pair sequence in the thymidilate synthase gene's 5′ UTR whereby the subject will exhibit the poorest response to administration of the TS directed drug if the subject's genotype is homozygous for a triple repeat of the tandemly repeated sequence, a less poor response to administration of the TS directed drug if the subject's genotype is heterozygous for a double repeat and a triple repeat of the tandemly repeated sequence, and the best response to administration of the TS directed drug if the subject's genotype is homozygous for a double repeat of the tandemly repeated sequence.

27. The method of claim 26 wherein the TS directed drug is a fluoropyrimidine.

28. The method of claim 27 wherein the fluoropyrimidine is 5-fluorouracil.

29. A method for determining an appropriate chemotherapeutic regimen to treat a cancer in a subject, the method comprising:
associating a genomic polymorphism of the subject with the effectiveness of a chemotherapeutic regimen.

30. The method of claim 29 wherein the method is used to select or reject a chemotherapeutic drug to treat the cancer.

31. A kit for use in screening for the effectiveness of TS directed drug therapy in human subjects.

32. The kit of claim 31 comprising:
all or some of the positive controls, negative controls, reagents, primers, sequencing markers, probes and antibodies for determining the presence or absence of the tandemly repeated 28 base-pair nucleic acid sequence that defines the genomic polymorphism in the 5′ UTR of the TS gene.

33. The kit of claim 31 wherein the kit components may be provided in solution or as a liquid dispersion or the like.

34. The kit of claim 31 comprising DNA tandemly repeated sequences that determine the type of genomic polymorphism of the TS gene in Tris-EDTA buffer solution preferably kept at 4° C.


SEQUENCE LISTING (7 sequences)

SEQ ID NO: 1 - DNA, 18 bp, Artificial Sequence
  Description of Artificial Sequence Synthetic oligonucleotide
taatacgactcacttata

SEQ ID NO: 2 - DNA, 25 bp, Artificial Sequence
  Description of Artificial Sequence Synthetic primer
gatgtgcgcaatcatgtaacgtgag

SEQ ID NO: 3 - DNA, 30 bp, Artificial Sequence
  Description of Artificial Sequence Synthetic primer
gggagaggagttgaccaactgcaaagagtg

SEQ ID NO: 4 - DNA, 28 bp, Artificial Sequence
  Description of Artificial Sequence Synthetic primer
gggagagcgggaaatcgtgcgtgacatt

SEQ ID NO: 5 - DNA, 24 bp, Artificial Sequence
  Description of Artificial Sequence Synthetic primer
gatggagttgaaggtagtttcgtg

SEQ ID NO: 6 - DNA, 20 bp, Artificial Sequence
  Description of Artificial Sequence Synthetic primer
gtggctcctgcgtttccccc

SEQ ID NO: 7 - DNA, 29 bp, Artificial Sequence
  Description of Artificial Sequence Synthetic primer
gctccgagccggccacaggcatggcgcgg